(19) United States
(12) Patent Application Publication
(10) Pub. No.: US 2025/0321166 A1
(43) Pub. Date: Oct. 16, 2025

(54) System, Method and Device for Preparing and Storing Biological Material

(71) Applicant: Presstine LLC, Dover, DE (US)

(72) Inventor: Kenny MANI, Gilbert, AZ (US)

(73) Assignee: Presstine LLC, Dover, DE (US)

(21) Appl. No.: 19/250,326

(22) Filed: Jun. 26, 2025

Related U.S. Application Data
(63) Continuation of application No. PCT/US2023/083322, filed on Dec. 11, 2023.
(60) Provisional application No. 63/477,388, filed on Dec. 28, 2022.

(51) Int. Cl. G01N 1/36; G01N 1/34; G01N 1/42

(52) U.S. Cl. CPC G01N 1/36; G01N 1/34; G01N 1/42

(57) ABSTRACT

A system, method for preserving biological samples is disclosed. The illustrative method includes providing a biological sample for preparation and storage, and sealing the biological sample with a seal satisfying one or more criteria. The one or more criteria can be based on properties of the biological sample to ensure biological compatibility. The method includes processing the sealed biological sample for further sealing with another seal, the other seal being based on one or more other criteria.

### CROSS-REFERENCE TO RELATED APPLICATION(S)

This application is a Continuation of PCT Application No. PCT/US2023/83322 filed on Dec. 11, 2023, which claims priority to U.S. Provisional Patent Application No. 63/477,388 filed on Dec. 28, 2022, the contents of which are incorporated herein by reference in their entirety.

### TECHNICAL FIELD

The following generally relates generally to preparing and storing biological material(s), for example to preserve such biological materials, and particularly to storing and preserving biological materials with a seal.

### BACKGROUND

Storage of biological materials or samples has not significantly changed in decades. A biological material or sample is typically extracted and deposited onto a petri dish or other type of storage device. The sample material can be sealed to prevent contamination. The sealed material can thereafter be transported in boxes or via other handling and/or logistical solutions.

As a result, existing systems can be vulnerable to failure due to a variety of circumstances. For example, existing systems can be vulnerable to failure in the event the seal breaks or malfunctions. Existing systems can also be vulnerable if related logistics solutions do not provide sufficient means to assess the fidelity of the biological sample. Existing systems can also be vulnerable in that the process may be difficult to automate; for example, the process may include the use of multiple perishable or nonuniform elements which can make automation difficult or complicated.

Existing systems can also be impractical. The sample containers can be difficult to stack for logistics purposes. The sample containers can also require special handling instructions, increasing the cost associated with shipping biological samples.

### SUMMARY

To address challenges in existing biological sample storage systems, a system and method for preparing and storing biological material, and a related seal are disclosed herein.

In one aspect, there is provided a method for preparing and storing biological samples. The method includes providing a biological sample, and sealing the biological sample with a seal satisfying one or more criteria. The one or more criteria are based on properties of the biological sample to ensure biological compatibility. The method includes processing the sealed biological sample for further sealing with another seal, the other seal being based on one or more other criteria.

In example embodiments, at least some of the seal or the other seal is reusable.

In example embodiments, the biological sample comprises two different biological samples of the same material, with each biological sample being separately sealed.

In example embodiments, processing the sealed biological sample for further sealing, or sealing the biological sample includes freezing the sealed biological sample. The sealed biological sample can be frozen to an ultra-low temperature.

In example embodiments, the sealed biological sample is processed for further sealing without exposing the sealed biological sample to additional contaminants.

In example embodiments, the seal is rigid and supports at least one of rapid warming for disinfecting and freezing for storage.

In example embodiments, the seal or the other seal is a multi-part seal.

In example embodiments, the method further includes storing the further sealed biological sample in a temperature maintaining mechanism.

In example embodiments, sealing the biological sample with a seal includes inserting a sample into a cavity in a body of the seal, sealing the cavity with a second part of the seal. Further sealing of the sealed biological sample includes sealing the sealed biological body with a second body complimentary to the body.

In example embodiments, the sealing and the further sealing includes disinfecting the seal or the other seal prior to, or after applying the seals.

In example embodiments, the sealing and the further sealing includes applying a tracking element to the seal or the other seal prior to, or after applying the seals.

In example embodiments, the sealing and the further sealing are at least in part automated.

In example embodiments, the seal or the other seal are made from one of a carbon based or boron-based nanotube.

In another aspect, a seal is disclosed. The seal includes, in a first configuration: a first part and a second part enclosing another seal, the first and second part being connected to hermetically seal the other seal within the first and second part. The seal includes, in a second configuration: the first part and second part including, respectively, a first and second connector for reusably connecting to one another to create a chamber for the other seal.

### BRIEF DESCRIPTION OF THE DRAWINGS

Embodiments will now be described with reference to the appended drawings wherein:

FIG. 1 illustrates an example system for preserving biological material.

FIG. 2 is a block diagram that illustrates a workflow for preserving biological material.

FIG. 3A is a perspective view of an example multi-part seal.

FIG. 3B is a perspective view of part of unassembled part of the example multi-part seal in FIG. 3A.

FIG. 4 is a block diagram of a workflow for further sealing a sealed sample.

FIGS. 5A, and 5B are block diagrams that illustrate methods of sealing a sample for preserving biological material.

FIG. 6 is a block diagram of an example method for tracking sealed samples.

### DETAILED DESCRIPTION

The following generally relates to systems, methods, and related seals for preserving biological materials. It is understood that any reference to the term “seal”, or “sealing,” is intended to convey, in a nonlimiting fashion, that the biological sample is closed, and/or shut off, and/or isolated from, an environment outside of the seal. Sealing, therefore, and as will be described further below, denotes a process by which a biological material is isolated from potential sources of contamination. It is understood that the term contamination means the inclusion of any biological material, or other material, that undermines any subsequent evaluation or testing of the biological material or changes the composition or properties of the biological material such that they are different from the properties of the biological material as intended to be sealed.

The disclosed system, method, and related seal relates to storage of biological materials, and illustratively a method which requires sealing a sample with a first and a second seal. The two seals can be responsive to different criteria, such that each seal responds to different challenges related to the environment in which the seal will be stored or manipulated. For example, the first seal can be selected to ensure biological compatibility (e.g., the seal will not interact with the seal), whereas the second seal can be selected to survive ultra-low temperature and high-pressure environments. In example embodiments, the first seal is such that it enables the biological sample to be frozen according to high pressure freezing principles, whereas the second seal can be applied to ensure that the first seal is not tampered with, and to interface with a tracking system.

The seals can be reusable. For example, the second, external seal can be reused between seals in the first instance, which can facilitate investment in more robust and durable seals and related tracking systems. The reusable seals can be made from, for example, carbon based or boron-based nanotubes. In example embodiments, at least the outer seal is made from a rigid material and has a geometry that facilitates stacking the samples for storage (e.g., the outer seal can be rigid and shaped to fit within a cold storage tank or rack within a freezer).

The disclosed system and method can also include or interact with a tracking system, where each of the seals includes a tracking element. With each seal including a tracking element, the system can enable more accurate tracking, and provide additional data to track the fidelity of transportation and storage procedures to determine sample viability. For example, the inner tracking element can be used to determine a lab, location, and treatment applied to the biological material in the first seal, and the second seal tracking element can include the particulars of the transportation process related to the sample. In another example embodiment, the two seals provide the same information (e.g., the outer seal is at least in part transparent, allowing for updating of the two tracking elements to include or refer to the same information). The tracking element can be etched into the seal, such that a comprehensive and robust tracking system can be implemented with reusable seals.

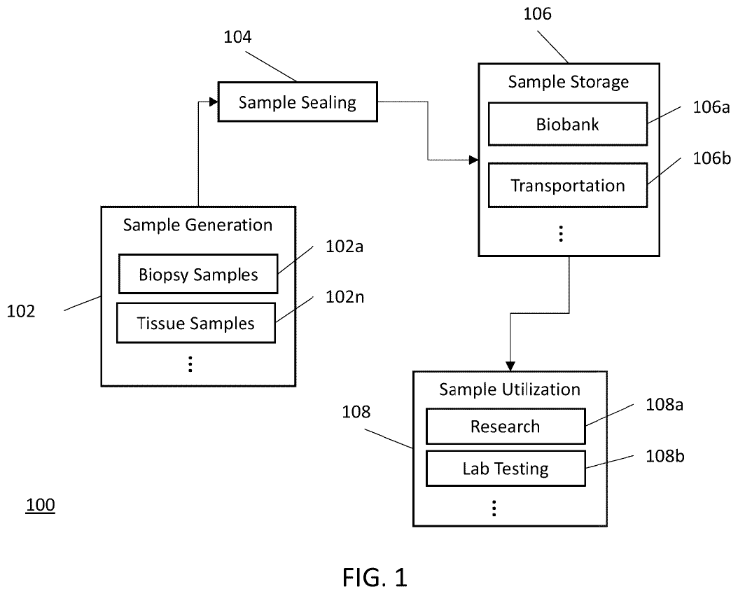

Referring now to FIG. 1, a system 100 for preserving biological material is shown. The example system 100 shown in FIG. 1 includes the following processes: sample generation 102, sample sealing 104, sample storage 106, and sample utilization 108.

In sample generation 102, a desired biological sample is retrieved, extracted, generated, etc. For example, in the example shown in FIG. 1, sample generation 102 can include biopsy extraction 102A, where the biological sample is actively extracted. In another example embodiment, the sample generation 102 can include tissue sample retrieval 102b, where the sample is provided (i.e., not actively extracted) by the source. It is understood that the examples are intended to be non-limiting, and that sample generation 102 can include, for example, generating a biological material in a synthetic environment, such as creating samples in a petri dish in a lab, etc. Biological material can include various substances, from proteins to tissues with animal, plants, or human origin.

In sample sealing 104, the sample generated during sample generation 102 is sealed, to prevent the sample from becoming contaminated. As will be discussed herein, the sealing process can include a variety of different types of processes, and a variety of combinations of the different types of processes. For example, sealing a sample can include freezing the sample to an ultralow temperature, in a pressurized chamber. Sealing the sample can include disinfecting the sealed sample, to inhibit any potential contamination that can occur because of the sealing process, or any subsequent handling.

In sample storage 106, the sealed sample is stored. Storage can take a variety of different forms and can involve a variety of different processes. In the example shown in FIG. 1, the seal sample may be stored in a bio bank 106a, or stored for transportation 106b (e.g., stored in a truck for transportation to biobank), and so forth. Sample storage 106 can include maintaining a variety of conditions to ensure sample integrity. For example, sample storage 106 can include maintaining the sealed sample in an ultra-low storage environment (e.g., within a cold storage container), a pressurized environment, etc.

In sample utilization 108, the sealed sample is utilized. A variety of different utilizations are contemplated. In the example shown in FIG. 1, the sealed sample is utilized by a research lab 108 for research purposes. Also shown in FIG. 1, the sealed sample can be utilized for diagnostic purposes, e.g., to perform testing on the sealed sample in a lab 108b (e.g., the sealed sample is tested to determine the presence of a form of cancer).

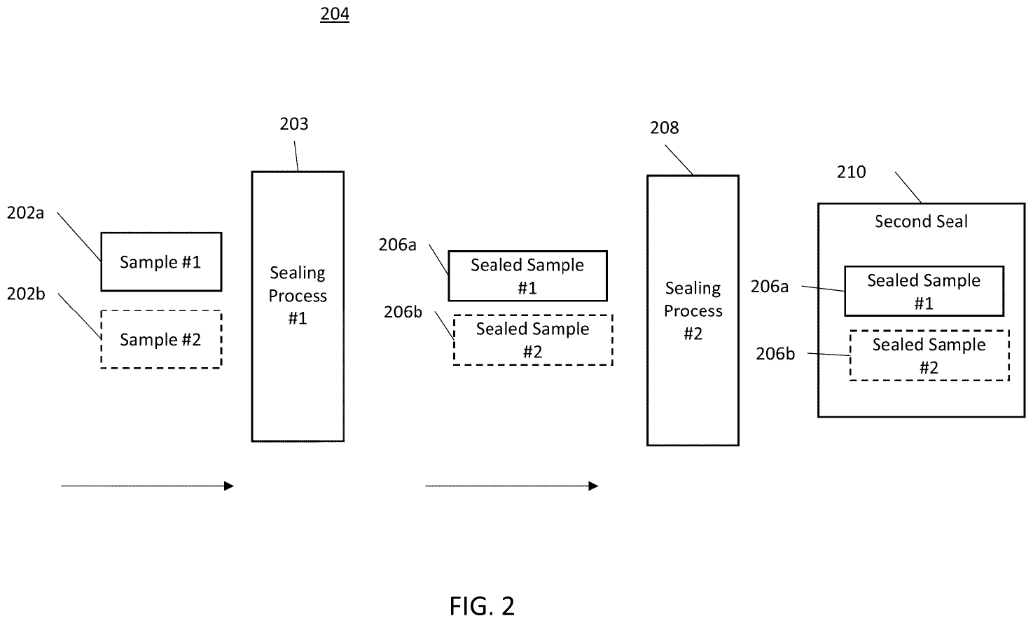

FIG. 2 is a block diagram that illustrates an example workflow 204 for preserving biological material.

One or more samples 202 are input into the sealing process 203. Two biological samples 202a, and 202b, are shown as going through the sample process 203 in FIG. 2. These samples are both sealed to provide for redundancy, to avoid negative outcomes associated with one of the sealed samples being compromised. For example, a biopsy sample 202 can be separated into two different, usable, amounts of material, and sealed in two separate sealed compartments 206a, 206b. The sealing process 203 can include sealing one or more samples 202, including sealing a plurality of unrelated samples 202 (e.g., samples from different biological sources are sealed with the sealing process 203, possibly simultaneously).

The sealing process 203 can include one or more of the following workflows in addition to the workflow of sealing the sample: disinfecting the container used to seal the sample 202, providing or connecting the container used to seal the sample 202 with a tracking element, etc.

The disinfecting can comprise disinfecting any part of the container used to seal the sample 202, prior to, during, or after sealing. In respect of disinfecting the container prior to sealing, various approaches are contemplated. For example, the container which is used to seal the sample 202 can be made of a material that can be raised to a temperature to kill all bacteria and other biological material residing on the container's surface, without impacting the containers performance. In another example, the container may be designed for relatively easy submersion in a disinfecting fluid or vapour (e.g., hydrogen peroxide) bath. In respect of disinfecting the container during the sealing process, various approaches are contemplated. For example, one half of a multi-part seal can be disinfecting immediately prior to being used to seal the sample 202. In respect of disinfecting the container after the sealing process, similar to disinfecting prior to the sealing, various approaches are contemplated.

In example embodiments, the container used to seal the sample 202 is sterile when provided to the sealing process 203. For example, the container may be disinfected prior to the process 203, and stored disinfected. The sealing process 203 can include retrieving the already disinfected container, and, for example, removing any packaging or other material used to maintain the disinfected container in a disinfected state.

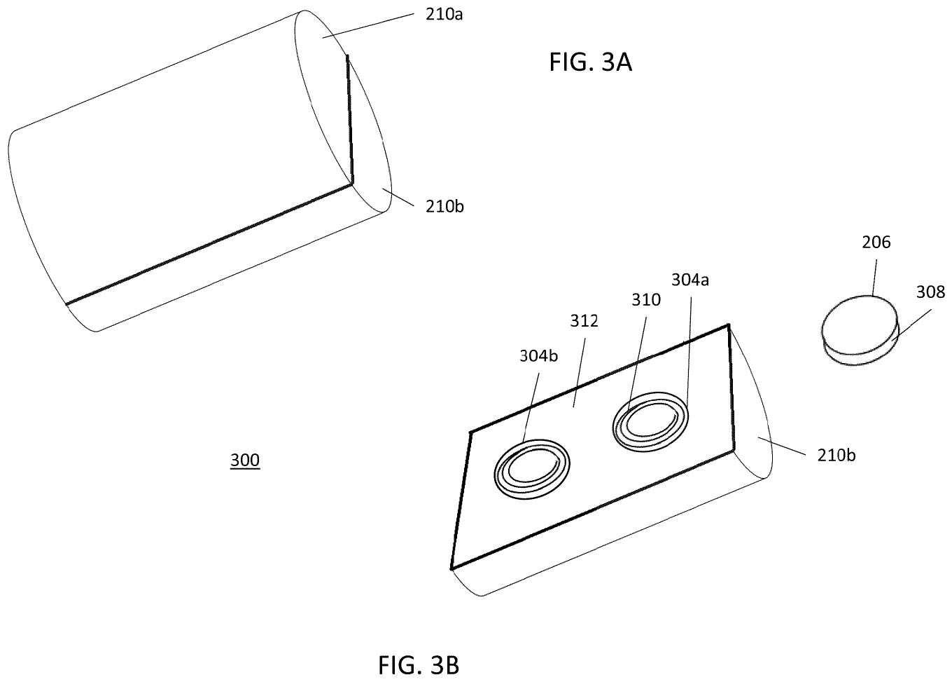

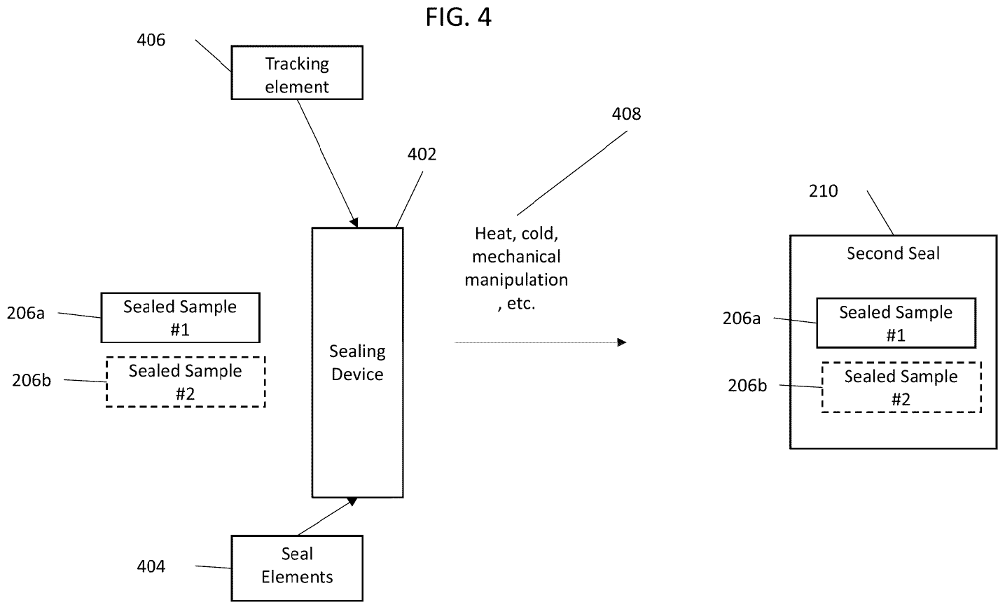

The sealing elements used to seal the sample (e.g., multi-part seal 300 of FIG. 3) can be made of a variety of suitable materials. The suitable material can be selected based on processes other than sealing, and can include, for example, materials which can withstanding disinfecting processes, freezing processes, etc., if required. The suitable material can also include a material with a minimum robustness. For example, the suitable material may be required to be able to resist certain impact or shear forces if a sealed sample is dropped. At the same time, the material may be required to be sufficiently rigid to enable stacking or retaining the biological material within the sample without undue stirring or other motion. Another consideration includes the suitability for adherence of biological materials (e.g., materials that interact with the biological material 202 may compromise the integrity of the sample). For example, suitable materials include boron, boron nitrate, or carbon nanotube-based materials, metals such as stainless steel (which may be preferred in the inner seal to facilitate rapid freezing of the sample), etc.

Similar to the disinfecting, the providing or connecting the sealed sample 206 with a tracking element can include a various of approaches to connect the tracking element, which can be applied at different stages of sealing (i.e., prior to, during, or after sealing).

Connect the tracking element can include, for example, one or more of the following: etching (e.g., where the container is etched or otherwise imprinted with a barcode, QR code, etc.), labelling on an exterior surface (e.g., wherein a QR code sticker, or barcode sticker, is applied to a surface), applying a color code to the surface of the container via colouring, etc. In example embodiments, the tracking element comprises measuring a set of characteristics of the container (e.g., weight, height, diameter, notch(es), shape, etc.). For example, containers having different weights can be used to seal samples 202 from different biological sources, to facilitate distinguishing between different sources.

Multiple tracking elements can be applied to the same sample 202. The different elements can track the same information (e.g., provide redundancy), or the different tracking elements can at least in part be associated with different information. For example, all tracking elements can be related to the type of sample, and the storage instructions, but each tracking element may be related to data about how that tracking element was applied.

The tracking elements can be applied or measured prior to, during, or after sealing. Tracking elements such as elements adhered to the container can be applied prior to the sealing 203, 208. In example embodiments, an imprinted element is provided on the container during manufacturing. Tracking elements can be applied during or approximately simultaneously during sealing. For example, where sealing includes a multi-part seal, sealing can include incorporating a part which is used for tracking. For example, the multi-part seal may be able to incorporate a plurality of labelled parts (e.g., or a tracking tag) to complete the seal, and connecting the tracking element can include selecting a particular labelled part.

The tracking elements can be connected to, or provided with the container, with the aid of a tracking system. For example, a computerized tracking system can be configured to catalogue all labelled parts in circulation and direct the individual or process sealing the sample 202 to use a particular labelled part (e.g., use bottom part 68795 for the multi-part seal). The tracking system can generate new tracking elements for each sealing process (on demand, or otherwise). For example, the tracking system can print QR labels to be applied during sealing. In another example, the tracking system can include a database to store information about the sample 202, the sealed sample 206, or parts of the process 203, 208. The database can store, for example, the weight of the sealed sample 206, and other characteristics, which can be used as confirmation of sample identify in addition to a barcode. The sample can store images of the sample 202, the sealed sample 206, or parts of the process 203, 208, for confirmation. In another example, the database stores additional details, such as when the sealing occurred, which lab, machine, or person performed the sealing, the type of sealing applied, the nature of the sample, the source of the sample, etc.

In example embodiments, the sealing process 203, 208 seals the sample(s) 202 in a re-sealable manner. For example, as alluded to above, the sealing process 203 includes separate, mutually interacting parts as part of a multipart seal. The multiple parts can be joined together in a variety of manners to generate a seal on the biological sample. For example, the separate parts can be joined via threading, an interference fit, etc. Various multi-part seals are contemplated, including seals including two or more parts. For example, the multi-part seal can include one or more clasp mechanisms to join two separate parts, which are complimented with a rubber O-ring to decrease the likelihood that the external environment can enter the volume to be sealed. In at least some embodiments, the multi-part seal includes one or more disposable elements or parts which cannot be reused. For example, sealing process 214 can include sealing a multi-part seal can with a sealant such as glue, or other material. The disposable material can be applied to the multi-part seal exclusively where the separate parts join, in proximity thereto, or on other parts of the multi-part seal.

At least some parts of a multi-part seal can be used in combination with parts other than the parts of the original multi-part seal. Various methods for ensuring re-usability and cross-functionality are contemplated. For example, referring now to FIGS. 3A and 3B, an example multi-part seal 300 is shown. The multi-part seal 300 includes two mating parts 210a, 210b. A plurality of mating parts 210 can be available during the sealing process, all manufactured or otherwise configurated to allow interchangeability. For example, the interchangeable parts can all be the same shape, or include elements that facilitate interchangeability (e.g., each part has threading that can receive various corresponding threading from a sealed sample 206).

In the embodiment shown in FIG. 3A, the mating parts 210a, 210b include recesses 304a, 304b for receiving sealed samples 206. The sealed sample 206 includes threading 308 that engages the complementary threading 310 within one of the recesses 304 of the part 210b. In example embodiments, the different recesses 204 include different threading to receive different types of sealed samples 206, which may facilitate differentiating between sealed samples 206. The part 210a has recesses (not shown), corresponding to the profile of the sealed sample 206 extending from the recess 304 when connected to the part 210b.

The parts 210a, 210b can be connected to seal the sealed sample 206 (e.g., as shown in FIG. 3A). In the shown embodiment, the parts 210a, 210b include cooperating surfaces which facilitate the parts coming together to form a seal around the secured sealed sample 206. The surfaces, such as the shown surface 312 of part 210b, can be substantially planar, to enable the two cooperating surfaces to fit with one another. In example embodiments, the parts 210a, 210b include other types of cooperating features. For example, part 210a can include recesses (not shown) which cooperate with projections (not shown) of part 210b, to facilitate alignment, or to prevent misalignment. The additional cooperating features can be particularly important at preventing incorrect assembly that can result from fatigue or inattentiveness of workers sealing the samples. For example, in an environment which processes many sealed samples, the cooperating features can ensure that the parts are put together in a preferred orientation (e.g., the part may be at least in part symmetrical, with the asymmetry being important) to account for fatigue and difficulty in visually delineating between parts.

Referring again to FIG. 2, the two parts 210a, 210b, can be sealed together via the sealing process 208. This disclosure contemplates various methods of sealing the already sealed samples 206, which methods can be similar to the sealing process 203. For example, the container used to seal the sealed sample 206 can be multi-part seal 300. In another example, the container used to seal the sealed sample(s) 206 can be similar to a jar with a lid (not shown), with the lid being used to seal the sealed sample 206 into the jar.

The sealing process 208 can include one or more of the following workflows in addition to the workflow of sealing the sample: freezing the sealed sample 206, disinfecting the container used to seal the sealed sample 206, providing or connecting the container used to seal the sealed sample 206 with a tracking element, etc.

Various processes for freezing the sealed sample 206 are contemplated. For example, the freezing can include freezing the sealed sample 206 in a freezer to low temperatures (e.g., between zero (0) and minus 20 degrees Celsius), ultra-low temperatures (e.g., below minus 80 degrees Celsius), or other temperatures (e.g., certain samples 206 may require storage below minus 200 degrees Celsius).

The freezing can include freezing the sealed sample 206 under high pressure. For example, the freezing can occur within a high pressure ultra low freezer. The freezing can occur in other than a high-pressure environment.

The freezing process can occur substantially immediately, or over time. For example, the freezing process can include inserting the sealed sample 206 into a freezer which incrementally reduces the temperature of the sealed sample 206. In at least some contemplated example embodiments, the freezing is achieved by immersing the sealed sample 206 into a freezing liquid, which liquid immediately or relatively rapidly reduces the temperature of the sealed sample 206.

Similar to the disinfecting in sealing process 203, the disinfecting in the sealing process 206 can comprise disinfecting any part of the container used to seal the sealed sample 206, prior to, during, or after sealing. Repeating the example from process 203, in an example embodiment, the container which is used to seal the sealed sample 206 may be made of the same material as the first container used to seal the sample, with that material able to endure raised temperatures to kill all bacteria and other biological material residing on the container's surface. In another example, the container may be designed for relatively easy submersion in a disinfecting fluid or vapour bath.

As with sealing process 203, the providing or connecting the sealed sample with a tracking element can include a various of approaches to connect the tracking element, which can be applied at different stages of sealing (i.e., prior to, during, or after sealing).

In example embodiments, a different tracking element is used in sealing process 208 as compared to sealing process 203. This may introduce a robustness as the different tracking elements may react differently to different environments, to prevent simultaneous failure. For example, the first seal may be an etched QR code which can survive a sealing process, whereas the tracking element in sealing process 208 can be less sensitive to cold temperatures as it is applied after the freezing. Similar to process 203, the tracking elements can be applied or measured prior to, during, or after sealing.

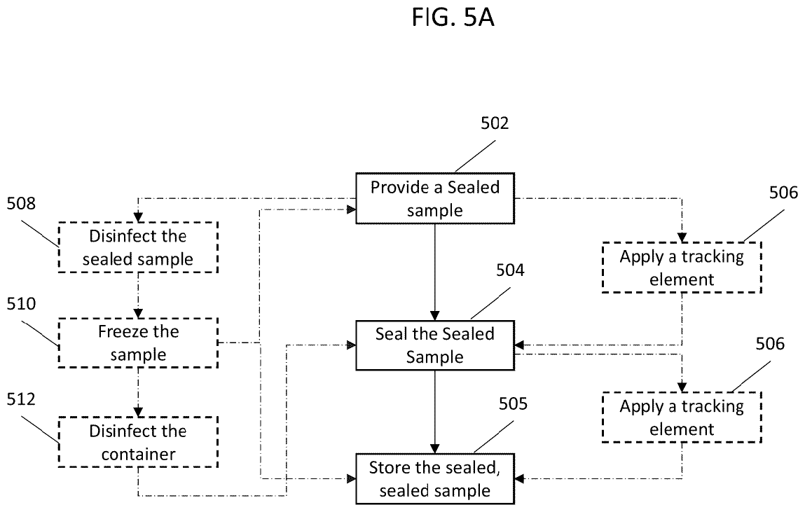

The sealing process 208 can be automated, or otherwise. Referring now to FIG. 4, the sealing process 208 is shown in greater detail.

The sealed sample 206 is inserted into the sealing device 402. In example embodiments, the sealed sample 206 is inserted into the device 402 via an automated process (e.g., via an automated mechanical arm, or inserted from a motorized tread, etc.), or the sealed sample 206 can be inserted into the device 402 other than in an automated fashion (the sealed sample can be placed into the device 402 by a person, etc.). In example embodiments, inserting the sealed sample 206 into the device 402 is a hybrid process including at least some automated processes. For example, a person may load a plurality of sealed samples for freezing into a feeder for the device 402 (not shown), and the device 402 can be configured to freeze samples within the feeder. In another example, the person can operate a mechanical arm.

One or more elements 404 required for the sealing process are input into the device 402. As with the sealed sample, the one or more elements 404 can be inserted in an automated, or other than automated, or hybrid fashion. In example embodiments, different elements of the elements 404 are inserted through separate processes. For example, the reusable parts may be provided by a first feeder mechanism, while a second process is used for perishable parts. In example embodiments, the elements 404 are assembled or utilized remotely, outside of the sealing device 402.

Optionally, one or more tracking elements 406 may be inserted or be otherwise provided to the sealing device 402. Similar to the one or more elements 404, the tracking elements 406 can be inserted in an automated, semi-automated, or other than automated fashion. In example embodiments, different elements of the elements 406 are inserted through separate processes.

The input tracking element 406 can data that is used by the sealing device 402 to perform sealing. For example, the tracking element 406 can include data that informs the device 402 of the sealing process to be applied, e.g., which reusable or perishable part to utilize, which tracking element to apply, which freezing process to apply, etc.

The sealing device 402, based on the elements 404, 406, and optionally based on additional input, applies the seal to the sealed sample 206. For example, the sealing device 402 may receive input to disinfect the sealed samples 206 prior to sealing them. In another example, the sealing device 402 can receive input which specifies the temperature to which the sealed sample 206 should be frozen. In example embodiments, the received input is received: via physical manipulation of the device 402 (e.g., button press, etc.), or via a wired or wireless connection to a controller (e.g., a remote control operates the device 402, for example where the device itself is hard to access or part of an automated processes), or via a wired or wireless connection to a computerized system (e.g., the input is received from a computerized system that tracks the sample 202).

As with sealing process 203, various methods of further sealing the sealed device 206 are contemplated. In example embodiments, as part of the sealing process 208, the sealing device 402 mechanically seals the sealed sample 206. For example, the sealing device 402 can seal the part 210b, with sealed samples 206 inserted therein, and placed with surface 312 facing up on a platform of the sealing device 402, with the part 210a in an automated fashion. Further particularizing the example, the device 402 can have the part 210b matingly receive the part 210a (e.g., by placing part 210a on top), and thereafter applying a sealant to the edge between the parts. In another example, now shown, the parts 210a, 210b may have partial threading that is complete when the parts 210a, 210b are joined, and an additional cap-like part is threaded onto the previously partial thread to seal the parts 210a, 210b. The sealing can be other than mechanical, relying upon bonding between parts of the multi-part seal (e.g., via heat-based fusing of two materials, gluing, etc.).

As alluded to above, the sealing device 402 can be a device which seals the sealed sample 206 and performs additional functions 408. For example, the sealing device 402 may also be an ultra-low temperature freezer, or a pressurized device, etc., to freeze or pressurize the sealed sample 206. The sealing device 402 can include or be composed of multiple devices. For example, the sealing device 402 can include a device for sealing the sealed sample 406, a device for disinfecting the sealed sample 206 (e.g., via ultraviolet, vapor washing, etc.) prior to its sealing, etc.

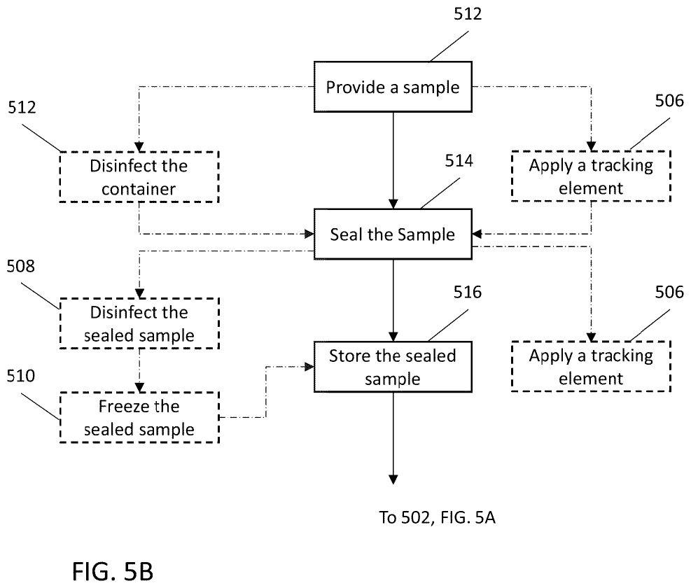

A variety of methods of sealing the sealed sample 206. Referring now to FIG. 5A, a block diagram of a method for preserving sealed biological samples is shown. The blocks in FIG. 5A, 5B shall be discussed with reference to the elements of the preceding FIGS. It is understood that these references are illustrative only, and not intended to be limiting.

At block 502, a sealed sample (e.g., sample 206) is provided. The providing can include retrieving the sealed sample 206 from storage in a biobank. In example embodiments, the sealed sample 206 is provided immediately after the sealing process which results in the sealed sample 206.

Optionally, at block 508, the sealed sample 206 is disinfected. Optionally, at block 510, the sealed sample is frozen. Optionally, at block 512, the container used to further seal the sealed sample 206 is disinfected. Combinations other than blocks 508, 510, and 512 in FIG. 5A are contemplated. For example, the method may not disinfect the sealed sample at block 508. Ordering other than the relative ordering of the blocks 508, 510, and 512 shown in FIG. 5A is also contemplated. For example, the sealed sample 206 may be frozen after the container used to further seal the sealed sample 206 is disinfected, to facilitate rapid sealing with the second seal.

At block 504, the sealed sample 206 is sealed (i.e., a second seal is applied) into twice sealed sample 210. The sample 202 can be sealed with a container satisfying one or more criteria based on properties of the sample to ensure biological compatibility. For example, the container can be selected to be inert relative to certain biological materials.

In example embodiments, multiple instances of block 510 can be included in a method for preserving biological material. For example, the sealed sample 206 can be frozen in a pressurized ultra-low temperature freezer, prior to the sealing in block 504, to preserve the structure of the biological sample. Thereafter, the second seal can be applied in block 504 sealing the frozen sealed sample 206. The second seal being frozen again can provide a buffer time which prevents the frozen sample 206 within the sample 210 from thawing. For example, the buffer time can be used to transport the sample 210 to the storage destination, or another insulating container for transport.

A tracking element 406 can be applied at any point in the method disclosed to this point. For example, as shown with blocks 506, the tracking element 406 can be applied to the sealed sample 206, prior to disinfection and freezing in blocks 508 and 510. In another example, the sealing can be applied to the twice sealed sample 210. In example embodiments, multiple tracking elements 406 are applied at different stages of the method. For example, a tracking element 406 can be applied to the sealed sample after disinfection (e.g., within the sealing device 402), and another tracking element 406 can be applied to the twice sealed sample 210, prior to disinfection.

FIGS. 5A and 5B together show a block diagram of a method for preserving biological samples.

At block 512, a biological sample 202 is provided.

At block 514, the sample 202 is sealed into sealed sample 206.

At block 516, the sealed sample 206 is stored, and thereafter provided as in block 502. In example embodiments, the sample 206 is immediately provided in block 502, and there is no separate storage block 516.

As with FIG. 5A, the various combinations of optional blocks 506, 508, 510, and 512 can be applied in between, prior to, or after the blocks 512, 514, or 516. For example, the container which is used to generate the sealed sample 206 can be disinfected prior to being used to generate the sealed sample, without additional disinfecting after the sealing process. In another example, the sealed sample 206 may be frozen, after which a tracking element 406 is applied, where the tracking element 406 is selected to withstand low temperature surfaces.

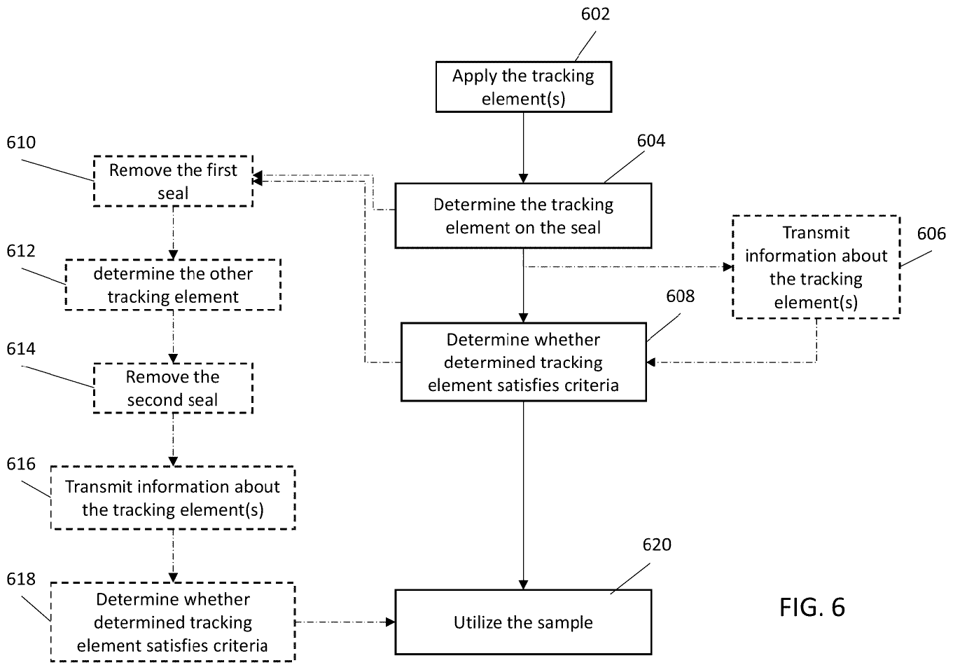

FIG. 6 shows a block diagram of a method for tracking preserved biological samples.

At block 602, the tracking elements 406 are applied to the sealed samples. As alluded to earlier above, a sealed sample can include multiple tracking elements 406 at different layers of the sealed sample.

Optionally, at block 606, information about the tracking elements 406 applied to the sample are transmitted to, for example, a database. In the example, the database may be a database operated by a party responsible for tracking and maintaining the fidelity of biological samples. The database may store data related to the tracking elements, the sample to which the tracking elements are applied, and other data. For example, database can store the type of tracking element applied, information to identify the tracking element (e.g., the barcode itself), when the tracking element was applied, to whom the sample belongs, which provider requested the sample, the sample destination, which sealing and other processes should apply to the sample, images of the tracking element, shipping preferences, storage requirements, etc. In example embodiments, where the tracking system is relatively rudimentary, for example, the tracking element can include all information required to identify the sample.

At block 604, the tracking element is determined. For example, the tracking element can be determined after the sealed sample has been transported to the destination where it will be utilized. Alternatively, the tracking element can be determined to determine an intended destination. Determining the tracking element can include the use of specialised equipment (e.g., a barcode scanner), or general-purpose devices (e.g., an application on a cellphone), and so forth.

Optionally, for example if a computerized system is used to track the sample, at block 606 the determined tracking event is transmitted to the computerized system. For example, the determined tracking element can be a barcode, and the barcode, and possibly identifying information about how the barcode was determined (which device, when, where, etc.) can be transmitted to the computerized system.

At block 608, the computerized system or a person can determine whether the determined tracking data satisfies certain criteria. The criteria can be related to a plurality of different properties related to the sample or to the tracking elements.

For example, the criteria can be based on the status of the tracking element (e.g., the tracking element has been compromised), the shipping method indicated by the tracking element (e.g., the sample was not shipped in a temperature-controlled manner, where that information is known), or the sample.

In respect of the sample, in an example embodiment, characteristics of the sample can be used as criteria to determiner whether information the computerized system has associated with the determined tracking information is correct. For example, one criterion can be a type of sealed sample (e.g., sealed in a tube-shaped seal), the weight of the sample, the type of sample (e.g., zoological, etc.). The computerized system can check the criteria to make sure that characteristics of the sample which are measured correspond with the records in the system.

If the criteria are satisfied, the sample can be used at block 620. Utilizing can include utilizing for diagnostic, research, or other purposes,

Optionally, in the even that the sample includes more than one tracking element (e.g., the twice sealed sample described herein), the first seal may be removed at block 610. In example embodiments, the seal is removed in a reusable manner.

At block 612, similar to block 604, the tracking element made visible after the removal of the seal in block 620 is determined. In example embodiments, block 610 is not performed as the first seal does not impede determining the second tracking element. For example, the second tracking element can be a cumulative weight, whereas the first tracking element can be a barcode. In example embodiments, the material of the first seal is sufficiently transparent to allow for determination of the other tracking element.

At block 614, the other seal is removed to allow for access to the biological sample. In example embodiments, block 614 includes removal of the seal only to the extent required to access the sample.

At block 616, the tracking information determined in block 612 is transmitted to the computerized system, and similar to block 608, at block 618, the transmitted information is used to determine whether the tracking element satisfies certain criteria. In example embodiments, block 618 is performed without a computerized system, as described in respect of block 608.

It is understood that this disclosure contemplates different the relative orders of the elements 610, 612, 614, 616, and 618, and different compositions of said elements. For example, the second seal can be removed in block 614 only after block 618 is satisfied. In another example, the method can include non-computerized methods which do not require transmission.

The disclosure at least in part relates to twice sealing biological samples. For clarity, and to avoid confusion, it is understood that the process(es) by which the two seals are formed, and the materials of the containers used to seal the materials, can be different as between the two seals. For example, the first seal can be a metal seal, which is disinfected only prior to the sample being deposited, and the second seal is a carbon nanotube material which is disinfected after the sealing process has been completed.

Furthermore, it is understood that the term seal includes a plurality of degrees of sealing, but preferably includes creating a seal that is compliant with biosafety level regulations (BSL), level two (2) or greater. The materials chosen and the sealing processes may be selected to enable long term storage (e.g., months or years), at ultra-low temperatures and otherwise.

For simplicity and clarity of illustration, where considered appropriate, reference numerals may be repeated among the figures to indicate corresponding or analogous elements. In addition, numerous specific details are set forth to provide a thorough understanding of the examples described herein. However, it will be understood by those of ordinary skill in the art that the examples described herein may be practiced without these specific details. In other instances, well-known methods, procedures, and components have not been described in detail so as not to obscure the examples described herein. Also, the description is not to be considered as limiting the scope of the examples described herein.

It will be appreciated that the examples and corresponding diagrams used herein are for illustrative purposes only. Different configurations and terminology can be used without departing from the principles expressed herein. For instance, components and modules can be added, deleted, modified, or arranged with differing connections without departing from these principles.

It will also be appreciated that any method steps exemplified herein can be at least in part automated, with the automation achieved via control of a machine or system with computer executable instructions which may include or otherwise have access to computer readable media such as storage media, computer storage media, or data storage devices (removable and/or non-removable) such as, for example, magnetic disks, optical disks, or tape. Computer storage media may include volatile and non-volatile, removable, and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the server or user's device, any component of or related thereto, etc., or accessible or connectable thereto. Any application or module herein described may be implemented using computer readable/executable instructions that may be stored or otherwise held by such computer readable media.

The steps or operations in the flow charts and diagrams described herein are just for example. There may be many variations to these steps or operations without departing from the principles discussed above. For instance, the steps may be performed in a differing order, or steps may be added, deleted, or modified.

Although the above principles have been described with reference to certain specific examples, various modifications thereof will be apparent to those skilled in the art as outlined in the appended claims.

1. A method for preparing and storing biological samples, the method comprising:
providing a biological sample;
sealing the biological sample with a seal satisfying one or more criteria, the one or more criteria based on properties of the biological sample to ensure biological compatibility; and
processing the sealed biological sample for further sealing with another seal, the other seal being based on one or more other criteria.

2. The method of claim 1, wherein at least some of the seal or the other seal is reusable.

3. The method of claim 1, wherein the biological sample comprises two different biological samples of the same material, with each biological sample being separately sealed.

4. The method of claim 1, wherein processing the sealed biological sample for further sealing, or sealing the biological sample comprises:
freezing the sealed biological sample.

5. The method of claim 4, wherein the sealed biological sample is frozen to an ultra-low temperature.

6. The method of claim 1, wherein the sealed biological sample is processed for further sealing without exposing the sealed biological sample to additional contaminants.

7. The method of claim 1, wherein the seal is rigid and supports at least one of rapid warming for disinfecting and freezing for storage.

8. The method of claim 1, wherein the seal or the other seal is a multi-part seal.

9. The method of claim 1, further comprising:
storing the further sealed biological sample in a temperature maintaining mechanism.

10. The method of claim 1, wherein sealing the biological sample with a seal comprises:
inserting a sample into a cavity in a body of the seal;
sealing the cavity with a second part of the seal;
and wherein further sealing the sealed biological sample comprises; and
sealing the sealed biological body with a second body complimentary to the body.

11. The method of claim 1, wherein the sealing and the further sealing comprise:
disinfecting the seal or the other seal prior to, or after applying the seals.

12. The method of claim 1, wherein the sealing and the further sealing comprise:
applying a tracking element to the seal or the other seal prior to, or after applying the seals.

13. The method of claim 1, wherein the sealing and the further sealing are at least in part automated.

14. The method of claim 1, wherein the seal or the other seal are made from one of a carbon based or boron-based nanotube.

15. A system for preparing and storing biological samples, the system comprising:
a biological sample; and
a first and second seal,
wherein the biological sample is sealed in the first seal, and the first seal is sealed within the second seal, and wherein the first seal is biologically compatible with the biological sample, and the second seal meets criteria associated with freezing processes.

16. The system of claim 15, wherein the first and second seal are rigid.

17. The system of claim 15, wherein the first or second seal comprise a tracking element imprinted thereon.

18. The system of claim 15, wherein the second seal is a multi-part seal with complimentary female and male parts.

19. The system of claim 15, wherein the second seal meets criteria for withstanding ultra-low temperature and high-pressure freezing procedures.

20. A seal comprising:
in a first configuration:
a first part and a second part enclosing another seal, the first and second part being connected to hermetically seal the other seal within the first and second part;
in a second configuration:
the first part and second part including, respectively, a first and second connector for reusably connecting to one another to create a chamber for the other seal.
